SEQUENCE LISTING (8 sequences)

SEQ ID NO: 1 - protein, 643 aa, Homo Sapiens [sequence omitted]

SEQ ID NO: 2 - DNA, 1932 bp, Homo Sapiens
atggggctcctgcccaagctcggcgcgtcccagggcagcgacacctctactagccgagccggccgctgtgcccgctcggtcttcggcaacattaaggtgtttgtgctctgccaaggcctcctgcagctctgccaactcctgtacagcgcctacttcaagagcagcctcaccaccattgagaagcgctttgggctctccagttcttcatcgggtctcatttccagcttgaatgagatcagcaatgccatcctcatcatctttgtcagctactttggcagccgggtgcaccgtccacgtctgattggcatcggaggtctcttcctggctgcaggtgccttcatcctcaccctcccacacttcctctccgagccctaccagtacaccttggccagcactgggaacaacagccgcttgcaggccgagctctgccagaagcattggcaggacctgcctcccagtaagtgccacagcaccacccagaacccccagaaggagaccagcagcatgtggggcctgatggtggttgcccagctgctggctggcatcgggacagtgcctattcagccatttgggatctcctatgtggatgacttctcagagcccagcaactcgcccctgtacatctccatcttatttgccatctctgtatttggaccggctttcgggtacctgctgggctctgtcatgctgcagatctttgtggactatggcagggtcaacacagctgcagttaacttggtcccgggtgacccccgatggattggagcctggtggctaggcctgctcatttcttcagctttattggttctcacctctttccccttttttttcttccctcgagcaatgcccataggagcaaagagggctcctgccacagcagatgaagcaaggaagttggaggaggccaagtcaagaggctccctggtggatttcattaaacggtttccatgcatctttctgaggctcctgatgaactcactcttcgtcctggtggtcctggcccagtgcaccttctcctccgtcattgctggcctctccaccttcctcaacaagttcctggagaagcagtatggcacctcagcagcctatgccaacttcctcattggtgctgtgaacctccctgctgcagccttggggatgctgtttggaggaatcctcatgaagcgctttgttttctctctacaagccattccccgcatagctaccaccatcatcaccatctccatgatcctttgtgttcctttgttcttcatgggatgctccaccccaactgtggccgaagtctacccccctagcacatcaagttctatacatccgcagtctcctgcctgccgcagggactgctcgtgcccagattctatcttccacccggtctgtggagacaatggaatcgagtacctctccccttgccatgccggctgcagcaacatcaacatgagctctgcaacctccaagcaactgatctatttgaactgcagctgtgtgaccgggggatccgcttcagcaaagacaggatcgtgccctgtcccctgtgcccacttcctgctcccggccatcttcctcatctccttcgtgtccctgatagcctgcatctcccacaaccccctctacatgatggttctgcgtgtggtgaaccaggaggaaaagtcatttgccatcggggtgcagttcttgttgatgcgcttgctggcctggctgccatctccagccctctatggcctcaccattgaccactcctgcatccggtggaactcgctgtgcttggggaggcgaggggcctgcgcctactatgacaacgatgctctccgagacaggtacctgggcctgcagatgggctacaaggcgctgggcatgctgctgctttgcttcatcagctggagggtgaagaagaacaaggagtacaacgtgcagaaggcggcaggcctcatctga

SEQ ID NO: 3 - DNA, 19 bp, Artificial Sequence
  Artificially synthesized
caatgcccataggagcaaa

SEQ ID NO: 4 - DNA, 20 bp, Artificial Sequence
  Artificially synthesized
cctccaacttccttgcttca

SEQ ID NO: 5 - DNA, 8 bp, Artificial Sequence
  Artificially synthesized
ctcctgcc

SEQ ID NO: 6 - DNA, 18 bp, Artificial Sequence
  Artificially synthesized
attggcaatgagcggttc

SEQ ID NO: 7 - DNA, 19 bp, Artificial Sequence
  Artificially synthesized
ggatgccacaggactccat

SEQ ID NO: 8 - DNA, 8 bp, Artificial Sequence
  Artificially synthesized
cttccagc

(19) United States
(12) Patent Application Publication
(10) Pub. No.: US 2022/0057405 A1
(43) Pub. Date: Feb. 24, 2022

(54) CELLS AND METHODS FOR EVALUATING THE ACTIVITY OF ORGANIC ANION TRANSPORTING POLYPEPTIDE 2A1 (OATP2A1)

(71) Applicant: ABSORPTION SYSTEMS LLC, Exton, PA (US)

(72) Inventors: Yuri BUKHTIYAROV, Exton, PA (US); Jibin LI, Exton, PA (US); Wei ZHANG, Exton, PA (US); Ismael HIDALGO, Exton, PA (US)

(21) Appl. No.: 17/433,322

(22) PCT Filed: Feb. 21, 2020
(86) PCT No.: PCT/US2020/019147

§ 371 Date: Aug. 24, 2021

Related U.S. Application Data
(60) Provisional application No. 62/809,920, filed on Feb. 25, 2019.

(51) Int. Cl. G01N 33/58; G01N 33/50; G01N 33/68

(52) U.S. Cl. CPC G01N 33/582; G01N 33/68; G01N 33/502

(57) ABSTRACT

Disclosed herein are cells overexpressing a human organic anion transporting polypeptide 2A1 (OATP2A1) and methods of measuring OATP2A1-mediated cellular uptake of a compound, methods of determining if a compound modulates OATP2A1-mediated activity, methods for determining if a cell expresses OATP2A1, as well as kits for carrying out the aforementioned methods.

### CROSS-REFERENCE TO RELATED APPLICATIONS

This application claims priority to U.S. provisional application No. 62/809,920, filed Feb. 25, 2019, the disclosure of which is incorporated herein by reference in its entirety.

### SEQUENCE LISTING

The instant application contains a Sequence Listing, which has been submitted electronically in ASCII format and is hereby incorporated by reference in its entirety. The Sequence Listing, which is labeled “104876.000036_SequenceListing.txt,” was created on Feb. 15, 2020, and is 10 KB.

### TECHNICAL FIELD

Disclosed herein are cells and methods for analyzing OATP2A1-mediated cellular uptake. In particular, the disclosed cells and methods can be used to screen compounds for their ability to modulate OATP2A1-mediated activity and to evaluate OAT2PA1-mediated activity and expression.

### BACKGROUND

Organic Anion Transporters (OATs) are plasma membrane proteins involved in cellular uptake and secretion of amphipathic drugs in tissues such as the intestine, liver, brain and kidneys. Organic Anion Transporting Polypeptide 2A1 (OATP2A1) is a solute carrier OAT, member 2A1, also known as the prostaglandin transporter (PGT). OATP2A1 is ubiquitously expressed in humans and its inhibition results in various pathological manifestations. OATP2A1 is essential for maintaining balanced regulation of the physiological processes mediated by prostaglandins via their re-uptake and metabolic degradation. Drugs modulating OATP2A1 activity may cause adverse effects manifesting themselves in reproductive processes, blood pressure and fever control as well as bone growth and resorption, enteropathy and perhaps other pathophysiological events resulting from dysregulation of prostaglandin signaling. Therefore, monitoring the effect of a drug on OATP2A1 activity and assessing the drug's susceptibility for active transport by OATP2A1 are useful for predicting a drug's safety and interactions with other drugs and nutrients.

### SUMMARY

Provided herein is a cell overexpressing a human organic anion transporting polypeptide 2A1 (OATP2A1), wherein the cell expresses at least 100 times more OATP2A1 mRNA compared to a control cell.

Also provided herein is a cell overexpressing a nucleic acid encoding a human organic anion transporting polypeptide 2A1 (OATP2A1), wherein the cell expresses at least 100 times more OATP2A1 mRNA compared to a control cell.

In some embodiments, the disclosed cell expresses at least 1000 times more OATP2A1 mRNA compared to the control cell. In some embodiments, the cell is transfected with an OATP2A1 nucleic acid. In other embodiments, the cell is stably transfected with the OATP2A1 nucleic acid. In some embodiments, the cell is a human embryonic kidney 293 (HEK293) cell. In other embodiments, the cell can be cultured for at least 20 passages without a significant reduction in OATP2A1 expression. In yet other embodiments, the cell further comprises a seminaphthorhodafluor (SNARF) substrate or a 6-carboxyfluorescein (6-CF) substrate. In some embodiments, wherein the SNARF substrate is a 5, 6-Carboxy-SNARF. In some embodiments, the 5, 6-Carboxy-SNARF is bound to the OATP2A1. In other embodiments, the cell has an OATP2A1-mediated activity that is at least 20 times greater than an OATP2A1-mediated activity of a control cell. In other embodiments, the cell has an OATP2A1-mediated uptake of a SNARF substrate that is at least 100 times greater than an OATP2A1-mediated uptake of a SNARF substrate in a control cell. In further embodiments, the OATP2A1 transporter-mediated activity or uptake is measured at a physiological pH ranging from about 6.5 to about 8.

Further provided herein is a cell overexpressing a human organic anion transporting polypeptide 2A1 (OATP2A1) and comprising a 5, 6-Carboxy-SNARF. In some embodiments, the cell expresses at least 100 times more OATP2A1 mRNA compared to a control cell.

Further provided herein is a method of measuring an organic anion transporting polypeptide 2A1 (OATP2A1)-mediated cellular uptake of a compound. The method comprises: (a) contacting a cell that overexpresses the OATP2A1 with the compound; and, (b) measuring the amount of the compound within the cell, wherein the amount of compound within the cell is indicative of the OATP2A1-mediated cellular uptake of the compound.

Also provided herein is a method of determining if a compound inhibits an OATP2A1. The method comprises: (a) contacting a cell that overexpresses the OATP2A1 with a fluorescent substrate; (b) contacting a cell that overexpresses the OATP2A1 with the compound and the fluorescent substrate; and (c) measuring an amount of the fluorescent substrate in the cell from step (a) and measuring an amount of the fluorescent substrate in the cell from step (b), wherein a decreased amount of the fluorescent substrate in the cell from step (b) indicates that the compound inhibits the OATP2A1.

Also provided herein is a method of determining if a compound inhibits an OATP2A1. The method comprises: (a) contacting a cell that expresses the OATP2A1 with a 5, 6-Carboxy-SNARF substrate; (b) contacting a cell that expresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a decreased amount of the 5, 6-Carboxy-SNARF substrate in the cell from step (b) indicates that the compound inhibits the OATP2A1.

Further provided herein is a method for determining if a compound modulates an OATP2A1-mediated activity. The method comprises: (a) contacting a cell that overexpresses the OATP2A1 with a fluorescent substrate; (b) contacting a cell that overexpresses the OATP2A1 with the compound and the fluorescent substrate; and (c) measuring an amount of the fluorescent substrate in the cell from step (a) and measuring an amount of the fluorescent substrate in the cell from step (b), wherein a change in the amount of the fluorescent substrate in the cell from step (b) indicates that the compound modulates the OATP2A1-mediated activity.

In some embodiments, an increase in the amount of the fluorescent substrate in the cell from step (b) indicates that the compound increases the OATP2A1-mediated activity and a decrease in the amount of the fluorescent substrate in the cell from step (b) indicates that the compound decreases the OATP2A1-mediated activity. In other embodiments, the fluorescent substrate comprises a seminaphthorhodafluor (SNARF) substrate, a 5, 6-Carboxy-SNARF substrate or a 6-carboxyfluorescein (6-CF) substrate.

Further provided herein is a method for determining if a compound modulates an OATP2A1-mediated activity. The method comprises: (a) contacting a cell that expresses the OATP2A1 with a 5, 6-Carboxy-SNARF substrate; (b) contacting a cell that expresses the OATP2A1 with the compound and the 5, 6-Carboxy-SNARF substrate; and (c) measuring an amount of the 5, 6-Carboxy-SNARF substrate in the cell from step a) and measuring an amount of the 5, 6-Carboxy-SNARF substrate in the cell from step b), wherein a change in the amount of the 5, 6-Carboxy-SNARF substrate in the cell from step b) indicates that the compound modulates the OATP2A1-mediated activity.

In some embodiments, an increase in the amount of the 5, 6-Carboxy-SNARF substrate in the cell from step (b) indicates that the compound increases the OATP2A1-mediated activity and a decrease in the amount of the 5, 6-Carboxy-SNARF substrate in the cell from step (b) indicates that the compound decreases the OATP2A1-mediated activity.

Further provided herein is a method of determining if a cell of interest expresses OATP2A1. The method comprises: (a) contacting the cell of interest with a 5, 6-Carboxy-SNARF substrate; (b) contacting a control cell that does not express OATP2A1 with the 5, 6-Carboxy-SNARF substrate; and (c) comparing an amount of the 5, 6-Carboxy-SNARF substrate within the cell of interest to an amount of the 5, 6-Carboxy-SNARF substrate within the control cell, wherein an increased amount of 5, 6-Carboxy-SNARF substrate in the cell of interest compared to the control cell indicates that the cell of interest expresses the OATP2A1.

In some embodiments of the various disclosed methods, the cell is the cell overexpressing an OATP2A1 as described above herein. In some embodiments, the cell is extracted from a mammalian tissue or human tissue.

In some embodiments, the disclosed compound comprises a new chemical entity (NCE), a nonsteroidal anti-inflammatory drug (NSAID), a modulator of eicosanoid receptor, a modulator of prostanoid receptor, or a modulator of prostaglandin transporter (PGT).

Additionally, provided herein is a kit comprising the cell overexpressing an OATP2A1 as described above herein; optionally a 5, 6-Carboxy-SNARF; and instructions for use.

A kit comprising a cell comprising a 5, 6-Carboxy-SNARF; and instructions for use is also provided.

In some embodiments, the instructions for use of the provided kits comprise instructions for measuring the ability of a compound to modulate the uptake of 5, 6-Carboxy-SNARF by an OATP2A1; providing an indication of whether or not the compound is a modulator or an inhibitor of OATP2A1; or both.

### BRIEF DESCRIPTION OF THE DRAWINGS

The summary, as well as the following detailed description, is further understood when read in conjunction with the appended drawings. For the purpose of illustrating the disclosed cells and methods, there are shown in the drawings exemplary embodiments of the disclosed cells and methods; however, the cells and methods are not limited to the specific embodiments disclosed. In the drawings:

FIG. 1 is a graph showing exemplary expression levels of OATP2A1 mRNA in the disclosed HEK293-OATP2A1 cell line.

FIG. 2 is an exemplary time course of OATP2A1-mediate uptake of 6-carboxyfluorescein (6-CF) by the HEK293-OATP2A1 cell line vs. the vector control cells.

FIG. 3A and FIG. 3B illustrate the structures of exemplary fluorescent substrates used in the experiments herein. FIG. 3A: 6-carboxyfluorescein (6-CF), and FIG. 3B: 5,6-Carboxy-Seminaphthorhodafluor (SNARF).

FIG. 4A and FIG. 4B depict an exemplary pH profile of OATP2A1-mediated uptake of a fluorescent substrate. FIG. 4A: pH profile of OATP2A1-mediated uptake of 5,6-Carboxy-SNARF. FIG. 4B: pH profile of OATP2A1-mediated uptake of the fluorescent substrate 6-CF.

FIG. 5 is a graph depicting an exemplary Michaelis-Menten curve for the OATP2A1-mediated uptake of 5,6-Carboxy-SNARF in the HEK293-OATP2A1 cell line.

FIG. 6A and FIG. 6B depict an exemplary time-course of the OATP2A1-mediated uptake of a fluorescent substrate. FIG. 6A: Time-course of the OATP2A1-mediated uptake of 5,6-Carboxy-SNARF vs. background fluorescence in the HEK293-OATP2A1 cell line and the control cell line at pH 7.5. FIG. 6B: Time-course of the OATP2A1-mediated uptake of 6-CF vs. background fluorescence in the HEK293-OATP2A1 cell line and the control cell line at pH 6.5.

### DETAILED DESCRIPTION OF ILLUSTRATIVE EMBODIMENTS

The disclosed cells and methods may be understood more readily by reference to the following detailed description taken in connection with the accompanying figures, which form a part of this disclosure. It is to be understood that the disclosed cells and methods are not limited to the specific cells and methods described and/or shown herein, and that the terminology used herein is for the purpose of describing particular embodiments by way of example only and is not intended to be limiting of the claimed cells and methods.

Unless specifically stated otherwise, any description as to a possible mechanism or mode of action or reason for improvement is meant to be illustrative only, and the disclosed cells and methods are not to be constrained by the correctness or incorrectness of any such suggested mechanism or mode of action or reason for improvement.

Throughout this disclosure, the descriptions refer to compositions (e.g. the disclosed cells) and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

### Definitions

Throughout this disclosure, various aspects of the invention may be presented in a range format. Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

When values are expressed as approximations, by use of the antecedent “about,” it will be understood that the particular value forms another embodiment. As used herein, the term “about” is to be understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

It is to be appreciated that certain features of the disclosed cells and methods which are, for clarity, described herein in the context of separate embodiments, may also be provided in combination in a single embodiment. Conversely, various features of the disclosed cells and methods that are, for brevity, described in the context of a single embodiment, may also be provided separately or in any subcombination.

As used herein, the singular forms “a,” “an,” and “the” include the plural referents unless context clearly indicates otherwise. Similarly, the word “or” is intended to include “and” unless the context clearly indicates otherwise.

As used herein, term “mammal” includes both human and non-human mammals.

As used herein the term “amount” refers to the abundance or quantity of a constituent in a mixture.

The term “comprising” is intended to include examples encompassed by the terms “consisting essentially of” and “consisting of”; similarly, the term “consisting essentially of” is intended to include examples encompassed by the term “consisting of.” As used herein, the terms “comprising,” “including,” “containing” and “characterized by” are exchangeable, inclusive, open-ended and do not exclude additional, unrecited elements or method steps.

The term “expression” as used herein is defined as the transcription and/or translation of a particular nucleotide sequence driven by its promoter.

The term “measuring,” as used herein, relates to determining an amount or concentration, preferably semi-quantitatively or quantitatively. Measuring can be done directly or indirectly.

Nucleic acid molecules useful in the methods of the invention include any nucleic acid molecule that encodes OATP2A1. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule.

Unless otherwise specified, a “nucleic acid encoding” includes all nucleotide sequences that are degenerate versions of each other and that encode the same amino acid sequence. The phrase nucleic acid sequence that encodes a protein or an RNA may also include introns to the extent that the nucleic acid sequence encoding the protein may in some version contain an intron(s).

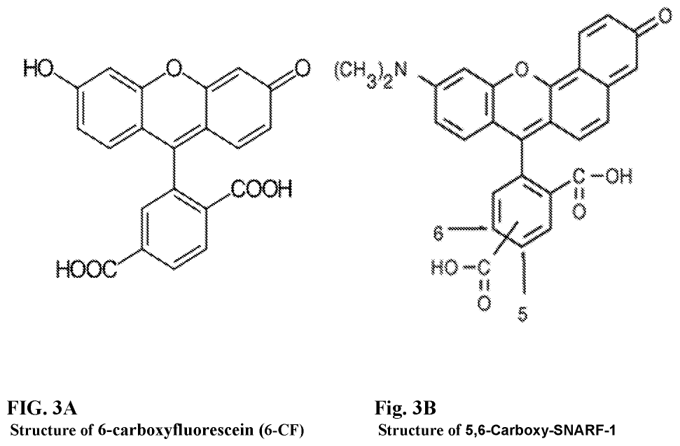

As used herein the terms “SNARF” refers to a seminaphthorhodafluor which is also known in the art as seminaphtharhodafluor or as seminaphtharhodafluorescein and is derived from rhodafluor (or rhodol) which is a molecule having the combined features of rhodamine and fluorescein. SNARF derivative 5, 6-Carboxy-SNARF or 5, 6-Carboxy-SNARF-1 are used interchangeably herein. In some embodiments, 5, 6-Carboxy-SNARF have a molecular formula of C27H19NO6 and a structure exemplified in FIG. 3B.

As used herein, the terms “reference”, or “control” are used interchangeably, and refer to a value that is used as a standard of comparison.

The term “transfected” or “transformed” or “transduced” as used herein refers to a process by which exogenous nucleic acid is transferred or introduced into the host cell. The exogenous nucleic acid can be, but is not limited to, a DNA, a cDNA, a mRNA or a miRNA. A “transfected” or “transformed” or “transduced” cell is one that has been transfected, transformed or transduced with exogenous nucleic acid. The cell includes the primary subject cell and its progeny. As used herein the term “uptake” refers to a transport from the outside of the cell to the inside of the cell including by OATP2A1-mediated transport across the membrane. Also, in some embodiments, the terms “substrate uptake”, “uptake”, “substrate transport” and “transport” can be used interchangeably when referring to the transport of the substrate within a cell.

Various terms relating to aspects of the description are used throughout the specification and claims. Such terms are to be given their ordinary meaning in the art unless otherwise indicated. Other specifically defined terms are to be construed in a manner consistent with the definitions provided herein.

### Description

OATP2A1 belongs to the 12-membrane-spanning superfamily of transporters and in humans is encoded by the solute carrier organic anion transporter family member 2A1 (SLCO2A1) gene. OATP2A1 is ubiquitously expressed especially in tissues abundant in prostaglandin synthesis, such as eye, lung, and kidney. OATP2A1 has a well-established role in mediating the distribution of prostanoids, such as prostaglandin E2 (PGE2), prostaglandin F2α (PGF2α), prostaglandin D2 (PGD2), and thromboxane B2 (TxB2). The transporter plays a key role in metabolic clearance of prostaglandins by actively uptaking them into the cell and then oxidatively inactivating by 15-ketoprostaglandin dehydrogenase. Physiological processes strongly mediated by prostaglandin signaling, such as reproductive functions, blood pressure, fever control, bone growth and resorption have been shown to depend heavily on OATP2A1 for maintaining delicate equilibrium between local production and disposition of prostaglandins. Due to ubiquitous expression of OATP2A1 in the body, pharmacokinetic properties of drugs and other compounds may be affected by their uptake by OATP2A1. The herein disclosed cells and methods can be used to measure OATP2A1-mediated uptake of compounds, analyze the ability of compounds to modulate the activity of OATP2A1, and determine if a cell of interest expresses OATP2A1, among other things.

Provided herein is a cell overexpressing a human OATP2A1 wherein the cell expresses at least 100 times more OATP2A1 mRNA compared to a control cell.

Also provided herein is a cell overexpressing a nucleic acid encoding a human OATP2A1, wherein the cell expresses at least 100 times more OATP2A1 mRNA compared to a control cell.

In some embodiments, the cell expresses at least 10 times, at least 50 times, at least 100 times, at least 200 times, at least 300 times, at least 500 times, at least 600 times, at least 700 times, at least 800 times, at least 900 times, at least 1000 times, at least 1100 times, at least 1200 times, at least 1300 times, at least 1400 times, at least 1500 times, at least 1600 times, at least 1700 times, at least 1800 times, at least 1900 times or at least 2000 times more OATP2A1 mRNA compared to the control cell. In some embodiments, the cell expresses at least 1000 times more OATP2A1 mRNA compared to the control cell.

The OATP2A1 can comprise the amino acid sequence of SEQ ID NO: 1 as provided in Table 5. In some embodiments, the OATP2A1 is the protein with accession number NP_005621.

The mRNA encoding OATP2A1 can comprise the nucleotide sequence of SEQ ID NO: 2 as provided in Table 5. In some embodiments, the mRNA encoding OATP2A1 is the SLCO2A1 mRNA with accession number NM_005630.2.

The cell can be transfected with a nucleic acid encoding OATP2A1. In some embodiments, for example, the cell is stably transfected with the nucleic acid encoding OATP2A1, wherein the transfection enhances the expression of OATP2A1. Methods for stable transfection of host cells with a gene of interest are well known in the art. These methods include, but are not limited to, the use a retroviral vector, electroporation, sonoporation, cell fusion, lipofection or the use of transfection reagents such as cationic liposome formulations (e.g. Lipofectamine®. Thermo Fisher Scientific). In some embodiments, the cell can be cultured for at least 5 passages, for at least 10 passages, for at least 15 passages, for at least 20 passages, for at least 25 passages, for at least 30 passages, for at least 35 passages, for at least 40 passages, for at least 45 passages, or for at least 50 passages without a significant reduction in OATP2A1 expression. In some embodiments, the cell can be cultured for more than 20 passages without a significant reduction in OATP2A1 expression. In some embodiments, the cell can be cultured for a time period ranging from about 24 hours to about 96 hours, from about 24 hours to about 72 hours, from about 24 hours to about 48 hours, or from about 24 hours to about 36 hours. In some embodiments, the cell can be cultured for about 48 hours. Any cell culture techniques, media and conditions known by a skilled artisan (e.g. incubation at 37° C. in 5% CO₂) could be used for the presently disclosed cells and methods.

In some embodiments, the cell has an OATP2A1-mediated activity that is at least 5 times, at least 10 times, at least 15 times, at least 20 times, at least 25 times, at least 30 times, at least 35 times, at least 40 times, at least 45 times or at least 50 times greater than an OATP2A1-mediated activity of a control cell. In some embodiments, the cell has an OATP2A1-mediated activity that is at least 20 times greater than an OATP2A1-mediated activity of a control cell.

Any mammalian cell line that can be manipulated to overexpress OATP2A1 can be used including, but not limited to, human embryonic kidney 293 (HEK293) cells or Chinese hamster ovarian (CHO) cells. In some aspects, the cell is a human embryonic kidney 293 (HEK293) cell. The control cell is preferably the same cell type as the cell overexpressing OATP2A1. In aspects where the cell is an HEK293 cell, for example, the control cell can be an HEK293 cell. In aspects where the cell is a CHO cell, the control cell can be a CHO cell.

Suitable control cells include, for example, cells transfected with a vector that does not comprise a nucleic acid encoding OATP2A1. In some instances, the control cell comprises a vector that favor the expression a fluorescent protein and/or a protein that provides resistance to an antibiotic such as but not limited to a Neomycin-resistance vector control HEK293 (HEK293-VC-Neo). In further embodiments, the control cell is any cell characterized by a normal level of OATP2A1 expression, including cells that have not been transfected or cells transfected with a vector that does not comprising the nucleic acid encoding OATP2A1 (e.g. empty vector).

The cell can further comprise a seminaphthorhodafluor (SNARF) substrate or a 6-carboxyfluorescein (6-CF) substrate (FIG. 3A). Any SNARF or SNARF derivative can be used in the cells and methods disclosed herein. In some embodiments, the SNARF substrate is a 5, 6-Carboxy-SNARF as shown in FIG. 3B and where the “5,6-Carboxy” can refer to the mixture of 5- and 6-carboxy substituted SNARF molecules. In some embodiments, the SNARF derivative is a fluoro-substituted derivative (e.g. SNARF™-5F 5-(and/or-6)-Carboxylic Acid (Thermo Fisher Scientific Cat. No. S23922)) or other chemical compounds within this class that comprises a fluorescent condensed ring structure of SNARF and a carboxylic group at either the 5- or 6-position on the phenyl ring. Without intending to be limiting, it is believed that, in addition to contributing to the anionic nature of the substrate for OATP2A1, the negative charge of the carboxylic group also renders the molecule cell-impermeable. Therefore, once the compound is actively transported inside the cell by OATP2A1, it remains inside the cell thanks of its negative charge and inability to passively diffuse out of the cell. In some aspects, the SNARF substrate or 6-CF substrate can be bound to the OATP2A1.

The cell can have an OATP2A1-mediated uptake of a SNARF substrate that is at least 10 times, at least 20 times, at least 30 times, at least 40 times, at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, at least 200 times, at least 210 times, at least 220 times, at least 230 times, at least 240 times, at least 250 times, at least 260 times, at least 270 times, at least 280 times, at least 290 times, at least 300 times, at least 400 times or at least 500 times more than an OATP2A1-mediated uptake of a SNARF substrate in a control cell. In some embodiments, the cell has an OATP2A1-mediated uptake of a SNARF substrate that is at least 100 times more than an OATP2A1-mediated uptake of a SNARF substrate in a control cell.

The OATP2A1-mediated activity or uptake can be measured at a pH ranging from about 6.5 to about 8. In some embodiments, the OATP2A1-mediated activity or uptake is measured at a pH ranging from about 5.5 to about 9, from about 6 to about 8.5, from about 6.5 to about 8 or from about 7 to about 7.5. In some embodiments, the OATP2A1-mediated activity or uptake is measured at a physiological pH that normally prevails in the human body, such as a pH of from about 7.35 to about 7.45.

Further disclosed herein are cells overexpressing a human organic anion transporting polypeptide 2A1 (OATP2A1) and comprising a 5, 6-Carboxy-SNARF. The 5, 6-Carboxy-SNARF substrate can be bound to the portion of the OATP2A1 located on the outer part of the cell membrane, the portion of the OATP2A1 located on the inner part of the cell membrane, or both the portion of the OATP2A1 located on the outer part and inner part of the cell membrane, such that none, some, or all of the 5, 6-Carboxy-SNARF substrate is present within the cell. For example, all of the 5, 6-Carboxy-SNARF substrate can be bound to the portion of the OATP2A1 located on the outer part of the cell membrane such that no substrate is present within the cell. Alternatively, all of the 5, 6-Carboxy-SNARF substrate can be bound to the portion of the OATP2A1 located on the inner part of the cell membrane such that all of the substrate is present within the cell. Alternatively, some of the 5, 6-Carboxy-SNARF substrate can be bound to the portion of the OATP2A1 located on the outer part of the cell membrane and some of the 5, 6-Carboxy-SNARF substrate can be bound to the portion of the OATP2A1 located on the inner part of the cell membrane such that substrate is present both on the cell membrane and within the cell. In other embodiments, the 5, 6-Carboxy-SNARF substrate can be within the cell and unbound from the OATP2A1 (such as in the cytoplasm of the cell). In some embodiments, the cell expresses at least 100 times more OATP2A1 mRNA compared to a control cell.

Also disclosed herein are methods of measuring an organic anion transporting polypeptide 2A1 (OATP2A1)-mediated cellular uptake of a compound. The disclosed methods comprise (a) contacting a cell that overexpresses the OATP2A1 with the compound; and, (b) measuring the amount of the compound within the cell, wherein the amount of the compound within the cell is indicative of the OATP2A1-mediated cellular uptake of the compound.

Also disclosed herein is the use of a cell overexpressing OATP2A1 in measuring the OATP2A1-mediated cellular uptake of a compound. The measuring can comprise contacting the cell with the compound and determining an amount of the compound within the cell, wherein the amount of the compound within the cell is indicative of the OATP2A1-mediated cellular uptake of the compound.

Disclosed herein are methods of determining if a compound inhibits an OATP2A1. The disclosed methods comprise: (a) contacting a cell that overexpresses the OATP2A1 with a fluorescent substrate; (b) contacting a cell that overexpresses the OATP2A1 with the compound and the fluorescent substrate; and (c) measuring an amount of the fluorescent substrate in the cell from step (a) and measuring an amount of the fluorescent substrate in the cell from step (b), wherein a decreased amount of the fluorescent substrate in the cell from step (b) indicates that the compound inhibits the OATP2A1.

Disclosed herein are methods of determining if a compound inhibits an OATP2A1. The disclosed methods comprise: (a) contacting a cell that expresses the OATP2A1 with a 5, 6-Carboxy-SNARF substrate; (b) contacting a cell that expresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a decreased amount of the 5, 6-Carboxy-SNARF substrate in the cell from step (b) indicates that the compound inhibits the OATP2A1.

Also disclosed herein are methods of determining if a compound inhibits an OATP2A1. The disclosed methods comprise: (a) contacting a cell that overexpresses the OATP2A1 with a 5, 6-Carboxy-SNARF substrate; (b) contacting a cell that overexpresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a decreased amount of the 5, 6-Carboxy-SNARF substrate in the cell from step (b) indicates that the compound inhibits the OATP2A1.

Further disclosed herein is the use of a cell overexpressing OATP2A1 for determining if a compound inhibits an OATP2A1. The determining can comprise (a) contacting a cell that overexpresses the OATP2A1 with a fluorescent substrate and (b) contacting a cell that overexpresses the OATP2A1 with the compound and the fluorescent substrate; and (c) measuring an amount of fluorescent substrate in the cell from step (a) and measuring an amount of fluorescent substrate in the cell from step (b), wherein a decreased amount of the fluorescent substrate in the cell from step (b) indicates that the compound inhibits the OATP2A1.

Disclosed herein is the use of a cell expressing OATP2A1 for determining if a compound inhibits an OATP2A1. The determining can comprise (a) contacting a cell that expresses the OATP2A1 with a 5, 6-Carboxy-SNARF substrate and (b) contacting a cell that expresses the OATP2A1 with the compound and the 5, 6-Carboxy-SNARF substrate; and (c) measuring an amount of 5, 6-Carboxy-SNARF substrate in the cell from step (a) and measuring an amount of 5, 6-Carboxy-SNARF substrate in the cell from step (b), wherein a decreased amount of the 5, 6-Carboxy-SNARF substrate in the cell from step (b) indicates that the compound inhibits the OATP2A1.

Also disclosed herein is the use of a cell overexpressing OATP2A1 for determining if a compound inhibits an OATP2A1. The determining can comprise (a) contacting a cell that overexpresses the OATP2A1 with a 5, 6-Carboxy-SNARF substrate; (b) contacting a cell that overexpresses the OATP2A1 with the compound and the 5, 6-Carboxy-SNARF substrate; and (c) measuring an amount of 5, 6-Carboxy-SNARF substrate in the cell from step (a) and measuring an amount of 5, 6-Carboxy-SNARF substrate in the cell from step (b), wherein a decreased amount of the 5, 6-Carboxy-SNARF substrate in the cell from step (b) indicates that the compound inhibits the OATP2A1.

In a further aspect, disclosed herein are methods for determining if a compound modulates an OATP2A1-mediated activity. The disclosed methods comprise: (a) contacting a cell that overexpresses the OATP2A1 with a fluorescent substrate; (b) contacting a cell that overexpresses the OATP2A1 with the compound and the fluorescent substrate; and (c) measuring an amount of the fluorescent substrate in the cell from step (a) and measuring an amount of the fluorescent substrate in the cell from step (b), wherein a change in the amount of the fluorescent substrate in the cell from step (b) indicates that the compound modulates the OATP2A1-mediated activity.

In some embodiments of the disclosed methods for determining if a compound modulates an OATP2A1-mediated activity, an increase in the amount of the fluorescent substrate in the cell from step (b) above indicates that the compound increases the OATP2A1-mediated activity and a decrease in the amount of the fluorescent substrate in the cell from step (b) above indicates that the compound decreases the OATP2A1-mediated activity.

In some embodiments of the disclosed methods, the fluorescent substrate comprises a seminaphthorhodafluor (SNARF) substrate, a 5, 6-Carboxy-SNARF substrate or a 6-carboxyfluorescein (6-CF) substrate.

In another aspect, the disclosed methods for determining if a compound modulates an OATP2A1-mediated activity can comprise: (a) contacting a cell that expresses the OATP2A1 with a 5, 6-Carboxy-SNARF substrate; (b) contacting a cell that expresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a change in the amount of the 5, 6-Carboxy-SNARF substrate in the cell from step (b) indicates that the compound modulates the OATP2A1-mediated activity.

In yet another aspect, the disclosed methods for determining if a compound modulates an OATP2A1-mediated activity can comprise: (a) contacting a cell that overexpresses the OATP2A1 with a 5, 6-Carboxy-SNARF substrate; (b) contacting a cell that overexpresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a change in the amount of the 5, 6-Carboxy-SNARF substrate in the cell from step (b) indicates that the compound modulates the OATP2A1-mediated activity.

In some embodiments of the disclosed methods for determining if a compound modulates an OATP2A1-mediated activity, an increase in the amount of the 5, 6-Carboxy-SNARF substrate in the cell from step (b) indicates that the compound increases the OATP2A1-mediated activity and a decrease in the amount of the 5, 6-Carboxy-SNARF substrate in the cell from step (b) indicates that the compound decreases the OATP2A1-mediated activity.

Also disclosed herein is the use of a cell that overexpresses OATP2A1 for determining if a compound modulates OATP2A1-mediated activity. The determining can comprise (a) contacting a cell that overexpresses the OATP2A1 with a fluorescent substrate; (b) contacting a cell that overexpresses the OATP2A1 with the compound and the fluorescent substrate; and (c) measuring an amount of the fluorescent substrate in the cell from step (a) and measuring an amount of the fluorescent substrate in the cell from step (b), wherein a change in the amount of the fluorescent substrate in the cell from step (b) indicates that the compound modulates the OATP2A1-mediated activity.

In some embodiments, an increase in the amount of the fluorescent substrate in the cell from step (b) above indicates that the compound increases the OATP2A1-mediated activity and a decrease in the amount of the fluorescent substrate in the cell from step (b) above indicates that the compound decreases the OATP2A1-mediated activity.

Disclosed herein is the use of a cell that expresses OATP2A1 for determining if a compound modulates OATP2A1-mediated activity. The determining can comprise (a) contacting a cell with a 5, 6-Carboxy-SNARF substrate; (b) contacting a cell that expresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a change in the amount of the 5, 6-Carboxy-SNARF substrate in the cell from step (b) indicates that the compound modulates the OATP2A1-mediated activity.

Further disclosed herein is the use of a cell that overexpresses OATP2A1 for determining if a compound modulates OATP2A1-mediated activity. The determining can comprise (a) contacting a cell with a 5, 6-Carboxy-SNARF substrate; (b) contacting a cell that overexpresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a change in the amount of the 5, 6-Carboxy-SNARF substrate in the cell from step (b) indicates that the compound modulates the OATP2A1-mediated activity.

In some embodiments, an increase in the amount of the 5, 6-Carboxy-SNARF substrate in the cell from step (b) above indicates that the compound increases the OATP2A1-mediated activity and a decrease in the amount of the 5, 6-Carboxy-SNARF substrate in the cell from step (b) above indicates that the compound decreases the OATP2A1-mediated activity.

Also provided herein are methods of determining if a cell of interest expresses OATP2A1. The methods comprise: (a) contacting the cell of interest with a 5, 6-Carboxy-SNARF substrate; (b) contacting a control cell that does not express OATP2A1 with the 5, 6-Carboxy-SNARF substrate; and (c) comparing an amount of the 5, 6-Carboxy-SNARF substrate within the cell of interest to an amount of the 5, 6-Carboxy-SNARF substrate within the control cell, wherein an increased amount of 5, 6-Carboxy-SNARF substrate in the cell of interest compared to the control cell indicates that the cell of interest expresses the OATP2A1.

The amount of 5,6-Carboxy-SNARF within the cell can correspond to a measurement of the OATP2A1 transporter activity which can be directly related to the amount of active OATP2A1 transporter protein expressed by the cells.

The cell of the methods disclosed herein can be a cell overexpressing the human OATP2A1 as described elsewhere herein.

In some embodiments, the cell disclosed herein can be extracted from a mammalian tissue or human tissue.

Suitable compounds for use in the methods disclosed herein include, but are not limited to, a new chemical entity (NCE), a nonsteroidal anti-inflammatory drug (NSAID), a modulator of eicosanoid receptor, a modulator of prostanoid receptor, or a modulator of prostaglandin transporter (PGT).

Also provided herein are kits comprising a cell overexpressing the human OATP2A1 as described elsewhere herein; optionally a 5, 6-Carboxy-SNARF; and instructions for use.

Further provided herein are kits comprising a cell comprising a 5, 6-Carboxy-SNARF; and instructions for use.

In some embodiments of the disclosed kits, the instructions comprise instructions for measuring the ability of a compound to modulate the uptake or the transport of 5, 6-Carboxy-SNARF by an OATP2A1; providing an indication of whether or not the compound is a modulator or an inhibitor of OATP2A1; or both.

Provided herein are methods for evaluating the safety or side effect(s) of a test drug. The provided methods comprise (a) contacting a cell overexpressing the human OATP2A1 with the drug; (b) measuring the ability of the drug to modulate the uptake of 5, 6-Carboxy-SNARF by OATP2A1; and (c) evaluating the safety or side effect of the test drug based upon the level of modulation of the uptake of the 5, 6-Carboxy-SNARF so measured as compared with the uptake of 5, 6-Carboxy-SNARF by OATP2A1 in the cell overexpressing the human OATP2A1 in the absence of the drug.

Provided herein are methods for predicting the interaction between a test drug and at least one other drug or at least one nutrient. The provided methods comprise: (a) contacting a cell overexpressing the human OATP2A1 with the test drug; (b) measuring the ability of the test drug to modulate the uptake of 5, 6-Carboxy-SNARF by OATP2A1; and (c) predicting the interaction between the test drug and the at least one other drug or the at least one nutrient based upon the level of modulation of the uptake of the 5, 6-Carboxy-SNARF so measured as compared with the uptake of 5, 6-Carboxy-SNARF by OATP2A1 in the cell overexpressing the human OATP2A1 in the absence of the test drug.

### ILLUSTRATIVE EMBODIMENTS

Provided here are illustrative embodiments of the disclosed technology. These embodiments are illustrative only and do not limit the scope of the present disclosure or of the claims attached hereto.

### Embodiment 1

A cell overexpressing a human organic anion transporting polypeptide 2A1 (OATP2A1), wherein the cell expresses at least 100 times more OATP2A1 mRNA compared to a control cell.

### Embodiment 2

A cell overexpressing a nucleic acid encoding a human organic anion transporting polypeptide 2A1 (OATP2A1), wherein the cell expresses at least 100 times more OATP2A1 mRNA compared to a control cell.

### Embodiment 3

The cell of embodiments 1 or 2, wherein the cell expresses at least 1000 times more OATP2A1 mRNA compared to the control cell.

### Embodiment 4

The cell of any one of the previous embodiments, wherein the cell is transfected with an OATP2A1 nucleic acid.

### Embodiment 5

The cell of embodiment 4, wherein the cell is stably transfected with the OATP2A1 nucleic acid.

### Embodiment 6

The cell of any one of the previous embodiments, wherein the cell is a human embryonic kidney 293 (HEK293) cell.

### Embodiment 7

The cell of any one of the previous embodiments, wherein the cell can be cultured for at least 20 passages without a significant reduction in OATP2A1 expression.

### Embodiment 8

The cell of any one of the previous embodiments, wherein the cell further comprises a seminaphthorhodafluor (SNARF) substrate or a 6-carboxyfluorescein (6-CF) substrate.

### Embodiment 9

The cell of embodiment 8, wherein the SNARF substrate is a 5, 6-Carboxy-SNARF.

### Embodiment 10

The cell of embodiment 9, wherein the 5, 6-Carboxy-SNARF is bound to the OATP2A1.

### Embodiment 11

The cell of any one of the previous embodiments, wherein the cell has an OATP2A1-mediated activity that is at least 20 times greater than an OATP2A1-mediated activity of a control cell.

### Embodiment 12

The cell of any one of the previous embodiments, wherein the cell has an OATP2A1-mediated uptake of a SNARF substrate that is at least 100 times greater than an OATP2A1-mediated uptake of a SNARF substrate in a control cell.

### Embodiment 13

The cell of embodiment 11 or embodiment 12, wherein the OATP2A1 transporter-mediated activity or uptake is measured at a physiological pH ranging from about 6.5 to about 8.

### Embodiment 14

A cell overexpressing a human organic anion transporting polypeptide 2A1 (OATP2A1) and comprising a 5, 6-Carboxy-SNARF.

### Embodiment 15

The cell of embodiment 14, wherein the cell expresses at least 100 times more OATP2A1 mRNA compared to a control cell.

### Embodiment 16

A method of measuring an organic anion transporting polypeptide 2A1 (OATP2A1)-mediated cellular uptake of a compound, the method comprising: contacting a cell that overexpresses the OATP2A1 with the compound; and, measuring the amount of the compound within the cell, wherein the amount of compound within the cell is indicative of the OATP2A1-mediated cellular uptake of the compound.

### Embodiment 17

A method of determining if a compound inhibits an OATP2A1, the method comprising: (a) contacting a cell that overexpresses the OATP2A1 with a fluorescent substrate; (b) contacting a cell that overexpresses the OATP2A1 with the compound and the fluorescent substrate; and (c) measuring an amount of the fluorescent substrate in the cell from step (a) and measuring an amount of the fluorescent substrate in the cell from step (b), wherein a decreased amount of the fluorescent substrate in the cell from step (b) indicates that the compound inhibits the OATP2A1.

### Embodiment 18

A method of determining if a compound inhibits an OATP2A1, the method comprising: (a) contacting a cell that expresses the OATP2A1 with a 5, 6-Carboxy-SNARF substrate; (b) contacting a cell that expresses the OATP2A1 with the compound and the 5, 6-Carboxy-SNARF substrate; and (c) measuring an amount of the 5, 6-Carboxy-SNARF substrate in the cell from step (a) and measuring an amount of the 5, 6-Carboxy-SNARF substrate in the cell from step (b), wherein a decreased amount of the 5, 6-Carboxy-SNARF substrate in the cell from step (b) indicates that the compound inhibits the OATP2A1.

### Embodiment 19

A method for determining if a compound modulates an OATP2A1-mediated activity, the method comprising: (a) contacting a cell that overexpresses the OATP2A1 with a fluorescent substrate; (b) contacting a cell that overexpresses the OATP2A1 with the compound and the fluorescent substrate; and (c) measuring an amount of the fluorescent substrate in the cell from step (a) and measuring an amount of the fluorescent substrate in the cell from step (b), wherein a change in the amount of the fluorescent substrate in the cell from step (b) indicates that the compound modulates the OATP2A1-mediated activity.

### Embodiment 20

The method of embodiment 19, wherein an increase in the amount of the fluorescent substrate in the cell from step (b) indicates that the compound increases the OATP2A1-mediated activity and a decrease in the amount of the fluorescent substrate in the cell from step (b) indicates that the compound decreases the OATP2A1-mediated activity.

### Embodiment 21

The method of any one of embodiments 17 and 19, wherein the fluorescent substrate comprises a seminaphthorhodafluor (SNARF) substrate, a 5, 6-Carboxy-SNARF substrate or a 6-carboxyfluorescein (6-CF) substrate.

### Embodiment 22

A method for determining if a compound modulates an OATP2A1-mediated activity, the method comprising: (a) contacting a cell that expresses the OATP2A1 with a 5, 6-Carboxy-SNARF substrate; (b) contacting a cell that expresses the OATP2A1 with the compound and the 5, 6-Carboxy-SNARF substrate; and (c) measuring an amount of the 5, 6-Carboxy-SNARF substrate in the cell from step a) and measuring an amount of the 5, 6-Carboxy-SNARF substrate in the cell from step b), wherein a change in the amount of the 5, 6-Carboxy-SNARF substrate in the cell from step b) indicates that the compound modulates the OATP2A1-mediated activity.

### Embodiment 23

The method of embodiment 22, wherein an increase in the amount of the 5, 6-Carboxy-SNARF substrate in the cell from step (b) indicates that the compound increases the OATP2A1-mediated activity and a decrease in the amount of the 5, 6-Carboxy-SNARF substrate in the cell from step (b) indicates that the compound decreases the OATP2A1-mediated activity.

### Embodiment 24

A method of determining if a cell of interest expresses OATP2A1, the method comprising: (a) contacting the cell of interest with a 5, 6-Carboxy-SNARF substrate; (b) contacting a control cell that does not express OATP2A1 with the 5, 6-Carboxy-SNARF substrate; and (c) comparing an amount of the 5, 6-Carboxy-SNARF substrate within the cell of interest to an amount of the 5, 6-Carboxy-SNARF substrate within the control cell, wherein an increased amount of 5, 6-Carboxy-SNARF substrate in the cell of interest compared to the control cell indicates that the cell of interest expresses the OATP2A1.

### Embodiment 25

The method of any one of embodiments 16, 17, 19, 20 and 24, wherein the cell is the cell of any one of embodiments 1-15.

### Embodiment 26

The method of any one of embodiments 16-24, wherein the cell is extracted from a mammalian tissue or human tissue.

### Embodiment 27

The method of any one of embodiments 16-23, wherein the compound comprises a new chemical entity (NCE), a nonsteroidal anti-inflammatory drug (NSAID), a modulator of eicosanoid receptor, a modulator of prostanoid receptor, or a modulator of prostaglandin transporter (PGT).

### Embodiment 28

A kit comprising: the cell of any one of embodiments 1-7 and 11-13; optionally a 5, 6-Carboxy-SNARF; and instructions for use.

### Embodiment 29

A kit comprising: a cell comprising a 5, 6-Carboxy-SNARF; and instructions for use.

### Embodiment 30

The kit of embodiment 28 or 29, wherein the instructions comprise instructions for measuring the ability of a compound to modulate the uptake of 5, 6-Carboxy-SNARF by an OATP2A1; providing an indication of whether or not the compound is a modulator or an inhibitor of OATP2A1; or both.

### Examples

The following examples are provided to further describe some of the embodiments disclosed herein. These Examples are provided for the purpose of illustration only and should in no way be construed as limiting the disclosed cells and methods, but rather should be construed to encompass any and all variations which become evident as a result of the teaching provided herein.

### Materials and Methods

Cells' Transfection

HEK293 cells were transfected with a vector comprising cDNA encoding OATP2A1 (“OATP2A1 expression plasmid”) (GeneCopoeia Cat. No. EX-Q0608-M02) using Lipofectamine® 3000 protocol (Thermo Fisher Scientific). The resulting HEK293-OATP2A1 cells were maintained in Dulbecco's Modified Eagle Medium (DMEM) supplemented with 1% L-glutamine and 25 mM HEPES (Thermo Fisher Scientific), 10% fetal bovine serum (FBS, Neuromics, Edina, Minn.), 100 U/mL of each penicillin and streptomycin (Thermo Fisher Scientific), and Geneticin® (G418) at 800 μg/mL (Thermo Fisher Scientific) for maintaining selective pressure. The cells resistant to G418 were subcloned using limiting dilution and the single clones were analyzed for OATP2A1 expression levels by QPCR, Western Blot analysis and OATP2A1 transporter activity.

Medium

HEK293-OATP2A1 and Neomycin-resistance vector control HEK293 (HEK293-VC-Neo) cells were maintained in Dulbecco's Modified Eagle Medium (DMEM) supplemented with 1% L-glutamine and 25 mM HEPES (Thermo Fisher Scientific), 10% fetal bovine serum (FBS, Neuromics, Edina, Minn.), and 100 U/mL of each penicillin and streptomycin (Thermo Fisher Scientific). Geneticin® (G418) at 800 μg/mL (Thermo Fisher Scientific) was also added to maintain selective pressure.

Cell Culture

Cells were cultured on Corning® CellBIND® 150 cm² flasks (Thomas Scientific). Cells were harvested by washing with 5 mL of Dulbecco's Phosphate Buffered Saline (DPBS, Thermo Fisher Scientific) and detaching them from the plastic surface with 3 mL of Trypsin-EDTA (Thermo Fisher Scientific). Cells were seeded in 24-well poly-D-Lysine coated (Biocoat®) plates (Becton Dickinson, Franklin Lakes, N.J.) at 2.5×10⁵ cells per well in 1 mL of the medium. Cells were maintained at 37° C. in 5% C02. Assays were typically run 48 hours after seeding. For cell shelf life evaluation, assays were run 1, 2, 3 and 4 days after seeding.

Real-Time Quantitative PCR

RNA Isolation—Total RNA was isolated from cultured cells using RNeasy Mini Kit (Qiagen, Valencia, Calif.) employing QIAcube (Qiagen) and following manufacturer's protocol. Qubit Fluorimeter (Thermo Fisher Scientific) was used with Quant-iT RNA Assay Kit (Thermo Fisher Scientific) for determination of RNA concentration according to manufacturer's protocol.

cDNA Synthesis—Approximately 1 μg of isolated total RNA was used for cDNA synthesis employing QuantiTect Reverse Transcription Kit (Qiagen) following the protocol supplied by Qiagen.

qPCR protocol—Gene expression analysis was performed by quantitative real-time PCR (qPCR) using LightCycler 480 System (Roche Molecular Systems, Indianapolis, Ind.). LightCycler® 480 Probes Master reaction mix (Roche) was used for setting up qPCR reactions with 0.01 μg of cDNA and the probe and primers at final concentrations of 0.1 μM and 0.5 μM, respectively. All samples were run in triplicate. All primers were custom-synthesized by Thermo Fisher Scientific and Universal Probe Library (UPL) probes were ordered from Sigma-Aldrich, St. Louis, Mo. (Table 2). The operating conditions for the qPCR reactions are listed in Table 3.

Data Analysis

The results were analyzed using LightCycler 480 Basic Software, Version 1.2. The fold-induction values were calculated vs. the housekeeping gene O-actin using the 2a method.

Cellular Uptake Assay with Fluorescent Substrate of the OATP2A1

Key reagents—6-Carboxyfluorescein (6-CF) was purchased from Toronto Research Chemicals (Cat. No. C178325); 5,6-carboxy-SNARF-1 (5,6-Carboxy-seminaphthorhodafluor-1) was obtained from Thermo Fisher Scientific (Cat. No. C1270).

All compounds were dissolved in DMSO at 10 mM concentration and kept at −20° C. as the DMSO stocks between the experiments.

Assay procedure—HEK293-OATP2A1 cells and the vector control HEK293-VC-Neo cells were plated at 250,000 cells per well on 24 well poly-D-Lysine coated (BioCoat™) plates (Corning) in complete DMEM medium and grown for 48 hrs at 37 C.

Cellular uptake of the fluorescent substrates 6-CF and 5,6-carboxy-SNARF-1 was assessed by incubating the HEK293-OATP2A1 and HEK293-VC-Neo cells with 10 μM 6-CF and 5 μM 5,6-carboxy-SNARF-1 for 15 and 20 min, correspondingly. For incubations with 6-CF, 1×HBSS pH 5.6 was used without pH adjustment. For the uptake of 5,6-carboxy-SNARF-1, the pH of 1×HBSS was adjusted to 7.4 with Bis-Tris/HCl. After the incubation, the media was aspirated, the cells were washed twice with ice-cold HBSS and lysed with 450 μL of the lysis buffer without the protease inhibitors. Two 150-μL samples from each well of the 24-well plates were taken and transferred into separate wells on black opaque 96-well Opti-plates (PerkinElmer). Fluorescence intensities were measured using λₑₓ 483(14) nm and λₑₘ 530(30) for 6-CF and λₑₓ 576(15) nm and λₑₘ 640(10) for 5,6-carboxy-SNARF-1 on a CLARIOstar® multimode microplate reader (BMG LABTECH). 20 μL of the samples used for fluorescence measurement were transferred into clear polystyrene flat-bottom 96-well plates (Thermo Fisher Scientific) and protein concentration was determined.

OATP2A1 Inhibition Assays

The list of OATP2A1 inhibitors used in this study is shown in Table 1. All compounds were acquired from Sigma-Aldrich as solid powders and 10 mM stocks were made either in DMSO or HBSS (see Table 1). Serial dilutions of the compounds were made using 3.162-fold (104) dilution factor either with DMSO or HBSS at the concentrations 1,000 higher than the final concentrations in the assay. These 1,000× solutions were further diluted 1:100 with HBSS in 2-mL polypropylene deep 96-well block (VWR) to make 10× working stock solutions of the inhibitors.

HEK293-OATP2A1 and HEK293-VC-Neo cells were cultured. The media was aspirated and 800 μL of HBSS was added to each well followed by the addition of 100 μL of the 10× working stock solutions of the inhibitors. The plates were incubated for 5 min at RT, and the assay was started by the addition of 100 μL of either 100 μM 6-CF or 50 μM 5,6-carboxy-SNARF-1 in HBSS.

TABLE 1
Inhibitors of OATP2A1 tested in the present study

| | | | Solvent for 10 |
|---|---|---|---|
| Compound | Supplier | Cat. No. | mM Stock |
| Suramin Sodium | Sigma-Aldrich | 574625-50MG | HBSS |
| Salt | | | |
| Pranlukast | Sigma-Aldrich | P0080-5MG | DMSO |
| hemihydrate | | | |
| Olmesartan | Sigma-Aldrich | SML1391-50MG | DMSO |
| medoxomil | | | |
| Zafirlukast | Sigma-Aldrich | Z4152-10MG | DMSO |
| Losartan | Sigma-Aldrich | 61188-100MG | DMSO |

Data Analysis

Concentrations of the fluorescent substrates of OATP2A1 in cell lysates were determined by measuring fluorescence intensities of the lysates and calculating concentrations of either 6-CF or 5,6-carboxy-SNARF-1 using linear calibration curves obtained from fluorescence intensities of the standard solutions of the fluorescent probes prepared in the same lysis buffer on the same 96-well assay plate. In order to account for differences in cell densities between the wells, protein concentration in the lysates was measured and used for calculating normalized uptake of the fluorescent substrates as follows:

$$Normalized Uptake ( pmole \text{/} mg ) = 1 \text{,} 000 \times \frac{{C}_{S} ( nM )}{{C}_{P} ( \frac{µg}{mL} )}$$

Using uninhibited normalized uptake values obtained with HEK293-OATP2A1 and HEK293-VC-Neo cells in 0.1% DMSO in HBSS as the high and low controls of the assay, the % Control and % Inhibition values were calculated as follows:

$$\% Control = 100 \% \times \frac{( N {U}_{T C} - N {U}_{H E K 293 - V C - N e o} )}{( N {U}_{H E K 293 - O A T P 2 A 1} - N {U}_{H E K 293 - V C - N e o} )}$$

$$\% Inhibition = 100 \% - \% Control$$

The IC₅₀ values were calculated from non-linear 4-parameter fit of the % Inhibition values vs. concentration of the inhibitors using GraphPad Prism 5 software. The same software was used for obtaining K_{M} and Vₘₐₓ values from a non-linear fit of the rate vs. substrate concentration fit into classic Michaelis-Menten equation:

$${V}_{t} = {V}_{max \times } \times \frac{S}{( {K}_{M} + S )}$$

Where:
C_{S} is the substrate concentration in cell lysate
C_{P} is the protein concentration in cell lysate
NU_{TC} is the normalized uptake of the substrate in the presence of test article or inhibitor
NU_{HEK293-VC-Neo} is the uninhibited (0.1% DMSO) normalized uptake by vector control cells
NU_{HEK293-OAM2A1} is the uninhibited (0.1% DMSO) normalized uptake by HEK293-OATP2A1 cells
Vₜ is the rate of the substrate uptake expressed as Normalized Uptake
Vₘₐₓ is the calculated maximal rate of the substrate uptake by HEK293-OATP2A1 cells
S is the concentration of the fluorescent substrate of OATP2A1
K_{M} is the calculated Michaelis constant.

TABLE 2
qPCR Primers and Probe Sequences

| | | | Sequence | | |
|---|---|---|---|---|---|
| | | | (5′ to 3′) | Cat. # | Lot # |
| OATP2A1 | Primers | OATP2A1F | CAATGCCCATAGGAGCAAA | | |
| | | | (SEQ ID NO: 3) | | |
| | | OATP2A1R | CCTCCAACTTCCTTGCTTCA | | |
| | | | (SEQ ID NO: 4) | | |
| | Probe | UPL #51 | CTCCTGCC | 04688481001 | 10336022 |
| | | | (SEQ ID NO: 5) | | |
| B-Actin | Primers | B-actinF | ATTGGCAATGAGCGGTTC | | |
| | | | (SEQ ID NO: 6) | | |
| | | B-actinR | GGATGCCACAGGACTCCAT | | |
| | | | (SEQ ID NO: 7) | | |
| | Probe | UPL #11 | CTTCCAGC | 04685105001 | 16842920 |
| | | | (SEQ ID NO: 8) | | |

TABLE 3
Conditionsfor qPCR

| qPCR Step | Number of Cycles | Temperature | Time | | Analysis |
|---|---|---|---|---|---|
| Pre-incubation | 1 | 95° C. | 5 | min | |
| Amplification | 45 | 95° C. | 10 | s | |
| | | 60° C. | 15 | s | |
| | | 72° C. | 1 | s | Single |
| Cooling | 1 | 40° C. | 5 | min | |

### Example 1: Characteristics of the HEK293-OATP2A1 Cell Line

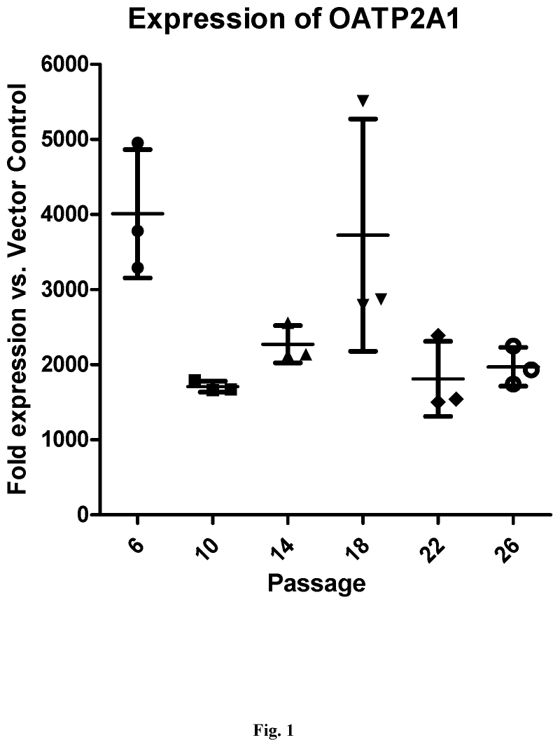

As shown in FIG. 1, the OATP2A1 expression plasmid was stably transfected as indicated by the expression levels of OATP2A1 mRNA in the HEK293-OATP2A1 cell line. Compared to the vector control cells, the HEK293-OATP2A1 cells maintain ≥1,000-fold higher OATP2A1 mRNA concentration over more than 20 passages as determined by QPCR.

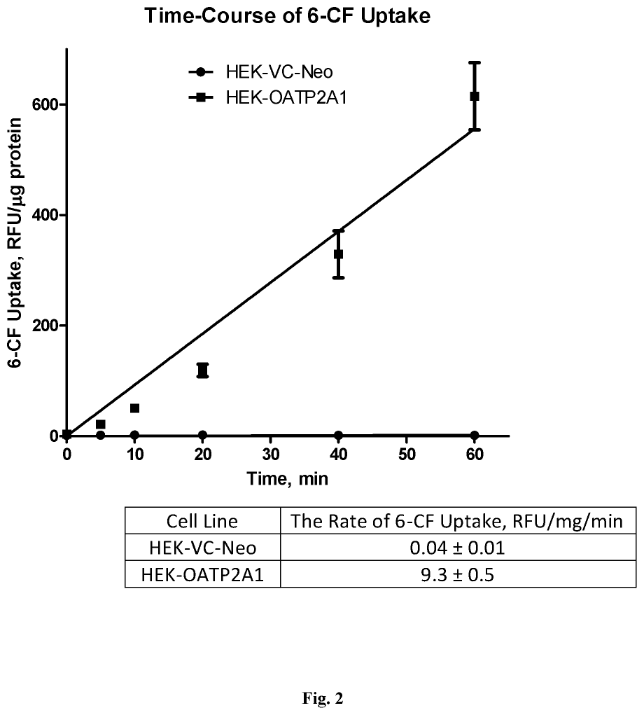

The graph and table from FIG. 2 depict the rates of OATP2A1-mediate uptake of 6-carboxyfluorescein (6-CF) by the HEK293-OATP2A1 cell line (“HEK-OATP2A1) vs. the vector control cells (HEK-VC-Neo). The HEK293-OATP2A1 cell line had ≥230-fold higher rate of 6-CF uptake compared to the control cells.

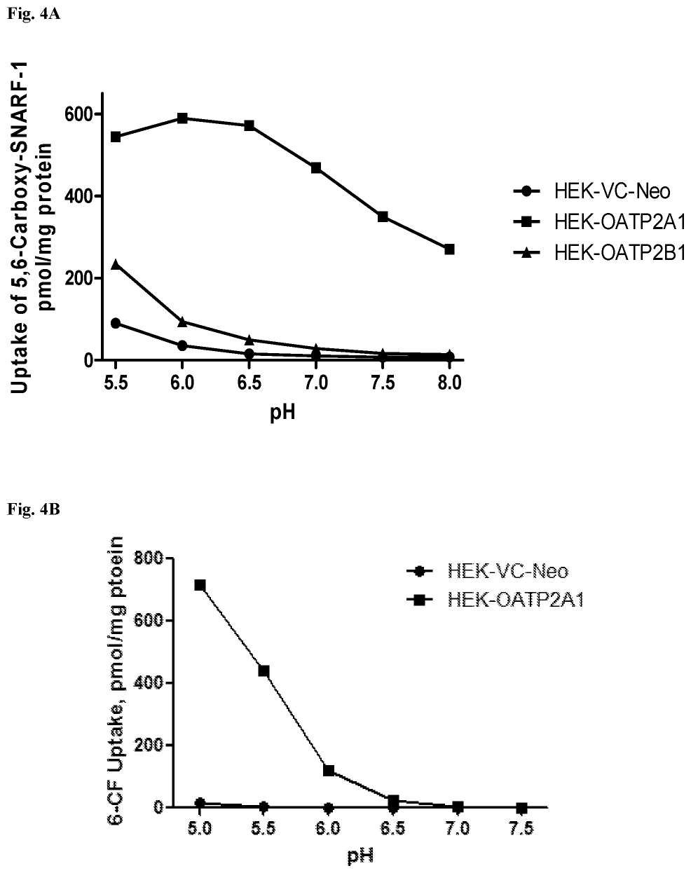

The pH profiles of OATP2A1 transporter activity with a fluorescent substrate are exemplified in FIGS. 4A and 4B. The signal to background (S:B) ratio exhibited by the HEK293-OATP2A1 cell line in 5,6-Carboxy-SNARF uptake assay ranged from 36 to 48 in the pH range from 6.5 to 8. A substantial assay window was observed at the physiological pH range of 7-7.5. Also, 5,6-Carboxy-SNARF was found to be specific for OATP2A1 vs. OATP2B1 transporters (FIG. 4A).

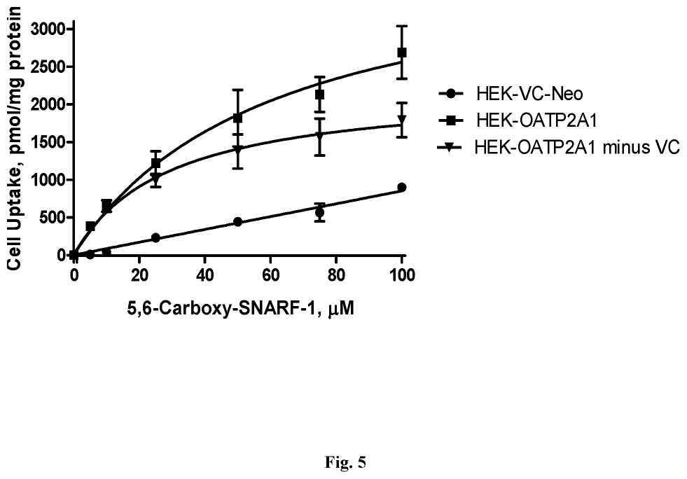

When the OATP2A1-mediated uptake of 5,6-Carboxy-SNARF in the HEK293-OATP2A1 cell line was assessed, a linear relationship between the concentration of 5,6-Carboxy-SNARF and fluorescence uptake by the vector control cells and a saturation exhibiting uptake by the HEK293-OATP2A1 cell line were observed (FIG. 5). The K_{M} for 5,6-Carboxy-SNARF for OATP2A1 was 29±9 μM.

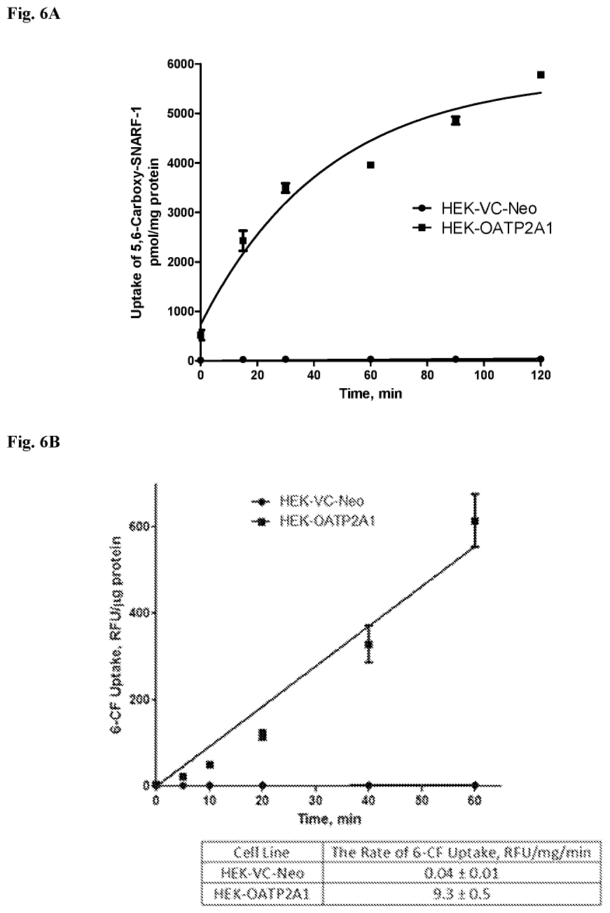

Time-courses of the OATP2A1 mediated uptake of a fluorescent substrate are exemplified in FIGS. 6A-6B. The rate of the uptake was linear for up to 30 min and provided substantial S:B ratio even after 15-20 min. The initial rates of the uptake over the first 30 min of the assay for HEK293-OATP2A1 vs. control cells were 99.1 and 0.7 pmol/mg/min, respectively. The uptake of 6-CF by the HEK293-OATP2A1 cell line is more than 1400 times higher than the rate of the 6-CF uptake by the control cells. The S:B for the HEK293-OATP2A1 cell line in the 5,6-Carboxy-SNARF uptake assay was 142 (FIG. 6A). The time-course in FIG. 6B represents the OATP2A1 mediated uptake of 6-CF vs. background fluorescence seen with the control cell line at pH 6.5. The uptake of 6-CF by the HEK293-OATP2A1 cell line was 233 times higher than the rate of the 6-CF uptake by the control cells (FIG. 6B).

Table 4 (below) lists the potencies of the known OATP2A1 inhibitors determined in the 6-CF and 5,6-Carboxy-SNARF uptake assay with the HEK293-OATP2A1 cell line. The IC₅₀ values reported in the literature (Kamo et al., J Pharm Sci, 2017. 106(9): p. 2483-2490) are also included for comparison. Despite the difference in pH and different passage of the HEK293-OATP2A1 cells, the IC₅₀ values were essentially the same between the 6-CF and 5,6-Carboxy-SNARF uptake assays and very similar to the published potencies of the OATP2A1 inhibitors.

TABLE 4
Potencies of the known OATP2A1 inhibitors in the assays of
cellular uptake of 6-CF and 5,6-Carboxy-SNARF with
HEK293-0ATP2A1 cells.

| | Literature (Kamo | | 5,6-Carboxy- |
|---|---|---|---|
| | et al., J Pharma | 6-CF uptake, | SNARF |
| Compound | Sci, 2017) | pH 5.5 | uptake, pH 7.4 |
| Suramin | 170 nM | 222 nM | 186 nM |
| Pranlukast | 550 nM | 142 nM | 148 nM |
| Olmesartan | 970 nM | 492 nM | 554 nM |
| Zafirlukast | 1,600 nM | N.A. | 1,114 nM |
| Losartan | 1,760 nM | 1,285 nM | 1,210 nM |

### Example 2: Significance of the OATP2A1 Overexpressing Cell Line

Robust methodology for measuring OATP2A1 transporter activity with fluorescent readout provides reliable and cost-efficient way for assessing NCEs and other compounds for potential modulation of the transporter function and its pharmacokinetic impact on absorption and distribution of the potential drug candidates. Significant overexpression of OATP2A1 in the HEK293-OATP2A1 cell line (>1,000-fold over the vector control cells on mRNA level) offered substantial signal to background (S:B) ratio in the functional OATP2A1 assay exceeding 40 at pH 7.5. The disclosed HEK293-OATP2A1 cell line exhibited robust expression of OATP2A1 (>1,000) and high S:B ratios (≥40) consistently over more than 20 passages.

The HEK293-OATP2A1 cell line was stable over multiple passages and over broad range of culture times (24-72 hrs) and the 5,6-Carboxy-SNARF offered an advanced methodology for measuring OATP2A1 activity.

### Example 3: Applications Using the HEK293-OATP2A1 Cell Line and/or the Fluorescent Substrate 5,6-Carboxy-SNARF

The disclosed methods employing either the disclosed HEK293-OATP2A1 cell line or the 5,6-Carboxy-SNARF or both can be used in large varieties of applications, including but not limited to the following:
Assessing cellular uptake of NCE's by the OATP2A1 using the HEK293-OATP2A1 cell line.
Evaluating NCE's for inhibitory potencies of OATP2A1 activity using a simple microplate-based assay monitoring accumulation of fluorescence in the HEK293-OATP2A1 cells incubated with 5,6-Carboxy-SNARF.
Screening large libraries for potential OATP2A1 modulators using a fluorescence microplate reader.
Determining expression level of OATP2A1 in different cells and tissues using the 5,6-Carboxy-SNARF in either microplate or flow-cytometry based format.
Designing diagnostic kits for all above mentioned applications utilizing the HEK293-OATP2A1 cell line and the 5,6-Carboxy-SNARF.

Table 5 below lists all the nucleic acid and amino acid sequences disclosed herein.

TABLE 5
Sequences

| SEQ | | |
|---|---|---|
| ID | Ref. | |
| NO | Name | Sequence |
| 1 | OATP2A1 | MGLLPKLGASQGSDTSTSRAGRCARSVFGNIKVFVLCQ |
| | | GLLQLCQLLYSAYFKSSLTTIEKRFGLSSSSSGLISSL |
| | | NEISNAILIIFVSYFGSRVHRPRLIGIGGLFLAAGAFI |
| | | LTLPHFLSEPYQYTLASTGNNSRLQAELCQKHWQDLPP |
| | | SKCHSTTQNPQKETSSMWGLMVVAQLLAGIGTVPIQPF |
| | | GISYVDDFSEPSNSPLYISILFAISVFGPAFGYLLGSV |
| | | MLQIFVDYGRVNTAAVNLVPGDPRWIGAWWLGLLISSA |
| | | LLVLTSFPFFFFPRAMPIGAKRAPATADEARKLEEAKS |
| | | RGSLVDFIKRFPCIFLRLLMNSLFVLVVLAQCTFSSVI |
| | | AGLSTFLNKFLEKQYGTSAAYANFLIGAVNLPAAALGM |
| | | LFGGILMKRFVFSLQAIPRIATTIITISMILCVPLFFM |
| | | GCSTPTVAEVYPPSTSSSIHPQSPACRRDCSCPDSIFH |
| | | PVCGDNGIEYLSPCHAGCSNINMSSATSKQUYLNCSCV |
| | | TGGSASAKTGSCPVPCAHFLLPAIFLISFVSLIACISH |
| | | NPLYMMVLRVVNQEEKSFAIGVQFLLMRLLAWLPSPAL |
| | | YGLTIDHSCIRWNSLCLGRRGACAYYDNDALRDRYLGL |
| | | QMGYKALGMLLLCFISWRVKKNKEYNVQKAAGLI |
| 2 | SLCO2A1 | ATGGGGCTCCTGCCCAAGCTCGGCGCGTCCCAGGGCAG |
| | | CGACACCTCTACTAGCCGAGCCGGCCGCTGTGCCCGCT |
| | | CGGTCTTCGGCAACATTAAGGTGTTTGTGCTCTGCCAA |
| | | GGCCTCCTGCAGCTCTGCCAACTCCTGTACAGCGCCTA |
| | | CTTCAAGAGCAGCCTCACCACCATTGAGAAGCGCTTTG |
| | | GGCTCTCCAGTTCTTCATCGGGTCTCATTTCCAGCTTG |
| | | AATGAGATCAGCAATGCCATCCTCATCATCTTTGTCAG |
| | | CTACTTTGGCAGCCGGGTGCACCGTCCACGTCTGATTG |
| | | GCATCGGAGGTCTCTTCCTGGCTGCAGGTGCCTTCATC |
| | | CTCACCCTCCCACACTTCCTCTCCGAGCCCTACCAGTA |
| | | CACCTTGGCCAGCACTGGGAACAACAGCCGCTTGCAGG |
| | | CCGAGCTCTGCCAGAAGCATTGGCAGGACCTGCCTCCC |
| | | AGTAAGTGCCACAGCACCACCCAGAACCCCCAGAAGGA |
| | | GACCAGCAGCATGTGGGGCCTGATGGTGGTTGCCCAGC |
| | | TGCTGGCTGGCATCGGGACAGTGCCTATTCAGCCATTT |
| | | GGGATCTCCTATGTGGATGACTTCTCAGAGCCCAGCAA |
| | | CTCGCCCCTGTACATCTCCATCTTATTTGCCATCTCTG |
| | | TATTTGGACCGGCTTTCGGGTACCTGCTGGGCTCTGTC |
| | | ATGCTGCAGATCTTTGTGGACTATGGCAGGGTCAACAC |
| | | AGCTGCAGTTAACTTGGTCCCGGGTGACCCCCGATGGA |
| | | TTGGAGCCTGGTGGCTAGGCCTGCTCATTTCTTCAGCT |
| | | TTATTGGTTCTCACCTCTTTCCCCTTTTTTTTCTTCCC |
| | | TCGAGCAATGCCCATAGGAGCAAAGAGGGCTCCTGCCA |
| | | CAGCAGATGAAGCAAGGAAGTTGGAGGAGGCCAAGTCA |
| | | AGAGGCTCCCTGGTGGATTTCATTAAACGGTTTCCATG |
| | | CATCTTTCTGAGGCTCCTGATGAACTCACTCTTCGTCC |
| | | TGGTGGTCCTGGCCCAGTGCACCTTCTCCTCCGTCATT |
| | | GCTGGCCTCTCCACCTTCCTCAACAAGTTCCTGGAGAA |
| | | GCAGTATGGCACCTCAGCAGCCTATGCCAACTTCCTCA |
| | | TTGGTGCTGTGAACCTCCCTGCTGCAGCCTTGGGGATG |
| | | CTGTTTGGAGGAATCCTCATGAAGCGCTTTGTTTTCTC |
| | | TCTACAAGCCATTCCCCGCATAGCTACCACCATCATCA |
| | | CCATCTCCATGATCCTTTGTGTTCCTTTGTTCTTCATG |
| | | GGATGCTCCACCCCAACTGTGGCCGAAGTCTACCCCCC |
| | | TAGCACATCAAGTTCTATACATCCGCAGTCTCCTGCCT |
| | | GCCGCAGGGACTGCTCGTGCCCAGATTCTATCTTCCAC |
| | | CCGGTCTGTGGAGACAATGGAATCGAGTACCTCTCCCC |
| | | TTGCCATGCCGGCTGCAGCAACATCAACATGAGCTCTG |
| | | CAACCTCCAAGCAACTGATCTATTTGAACTGCAGCTGT |
| | | GTGACCGGGGGATCCGCTTCAGCAAAGACAGGATCGTG |
| | | CCCTGTCCCCTGTGCCCACTTCCTGCTCCCGGCCATCT |
| | | TCCTCATCTCCTTCGTGTCCCTGATAGCCTGCATCTCC |
| | | CACAACCCCCTCTACATGATGGTTCTGCGTGTGGTGAA |
| | | CCAGGAGGAAAAGTCATTTGCCATCGGGGTGCAGTTCT |
| | | TGTTGATGCGCTTGCTGGCCTGGCTGCCATCTCCAGCC |
| | | CTCTATGGCCTCACCATTGACCACTCCTGCATCCGGTG |
| | | GAACTCGCTGTGCTTGGGGAGGCGAGGGGCCTGCGCCT |
| | | ACTATGACAACGATGCTCTCCGAGACAGGTACCTGGGC |
| | | CTGCAGATGGGCTACAAGGCGCTGGGCATGCTGCTGCT |
| | | TTGCTTCATCAGCTGGAGGGTGAAGAAGAACAAGGAGT |
| | | ACAACGTGCAGAAGGCGGCAGGCCTCATCTGA |
| 3 | OATP2A1F | CAATGCCCATAGGAGCAAA |
| 4 | OATP2A1R | CCTCCAACTTCCTTGCTTCA |
| 5 | UPL No51 | CTCCTGCC |
| 6 | B-actinF | ATTGGCAATGAGCGGTTC |
| 7 | B-actinR | GGATGCCACAGGACTCCAT |
| 8 | UPL No11 | CTTCCAGC |

The disclosures of each and every patent, patent application, and publication cited herein are hereby incorporated herein by reference in their entirety.

Those skilled in the art will appreciate that numerous changes and modifications can be made to the preferred embodiments of the disclosed cells and methods and that such changes and modifications can be made without departing from the spirit of the invention. It is, therefore, intended that the appended claims cover all such equivalent variations as fall within the true spirit and scope of the invention.

1. A cell overexpressing a human organic anion transporting polypeptide 2A1 (OATP2A1), wherein the cell expresses at least 100 times more OATP2A1 mRNA compared to a control cell.

2. (canceled)

3. The cell of claim 1, wherein the cell expresses at least 1000 times more OATP2A1 mRNA compared to the control cell.

4. The cell of claim 1, wherein the cell is transfected with an OATP2A1 nucleic acid.

5. The cell of claim 4, wherein the cell is stably transfected with the OATP2A1 nucleic acid.

6. The cell of claim 1, wherein the cell is a human embryonic kidney 293 (HEK293) cell.

7. The cell of claim 1, wherein the cell can be cultured for at least 20 passages without a significant reduction in OATP2A1 expression.

8. The cell of claim 1, wherein the cell further comprises a seminaphthorhodafluor (SNARF) substrate or a 6-carboxyfluorescein (6-CF) substrate.

9. The cell of claim 8, wherein the SNARF substrate is a 5, 6-Carboxy-SNARF.

10. The cell of claim 9, wherein the 5, 6-Carboxy-SNARF is bound to the OATP2A1.

11. The cell of claim 1, wherein the cell has an OATP2A1-mediated activity that is at least 20 times greater than an OATP2A1-mediated activity of a control cell.

12. The cell of claim 1, wherein the cell has an OATP2A1-mediated uptake of a SNARF substrate that is at least 100 times greater than an OATP2A1-mediated uptake of a SNARF substrate in a control cell.

13. The cell of claim 11, wherein the OATP2A1 transporter-mediated activity or uptake is measured at a physiological pH ranging from about 6.5 to about 8.

14.-15. (canceled)

16. A method of measuring an organic anion transporting polypeptide 2A1 (OATP2A1)-mediated cellular uptake of a compound, the method comprising:
a) contacting a cell that overexpresses the OATP2A1 with the compound; and,
b) measuring the amount of the compound within the cell, wherein the amount of compound within the cell is indicative of the OATP2A1-mediated cellular uptake of the compound.

17. A method of determining if a compound inhibits an OATP2A1 or modulates an OATP2A1 mediated activity, the method comprising:
a) contacting a cell that overexpresses the OATP2A1 with a fluorescent substrate;
b) contacting a cell that overexpresses the OATP2A1 with the compound and the fluorescent substrate; and
c) measuring an amount of the fluorescent substrate in the cell from step (a) and measuring an amount of the fluorescent substrate in the cell from step (b), wherein a decreased amount or a changed amount of the fluorescent substrate in the cell from step (b) indicates that the compound inhibits the OATP2A1, or that the compound modulates the OATP2A1-mediated activity.

18.-19. (canceled)

20. The method of claim 17, wherein an increase in the amount of the fluorescent substrate in the cell from step (b) indicates that the compound increases the OATP2A1-mediated activity and a decrease in the amount of the fluorescent substrate in the cell from step (b) indicates that the compound decreases the OATP2A1-mediated activity.

21. The method of claim 17, wherein the fluorescent substrate comprises a seminaphthorhodafluor (SNARF) substrate, a 5, 6-Carboxy-SNARF substrate or a 6-carboxyfluorescein (6-CF) substrate.

22.-23. (canceled)

24. A method of determining if a cell of interest expresses OATP2A1, the method comprising:
a) contacting the cell of interest with a 5, 6-Carboxy-SNARF substrate;
b) contacting a control cell that does not express OATP2A1 with the 5, 6-Carboxy-SNARF substrate; and
c) comparing an amount of the 5, 6-Carboxy-SNARF substrate within the cell of interest to an amount of the 5, 6-Carboxy-SNARF substrate within the control cell, wherein an increased amount of 5, 6-Carboxy-SNARF substrate in the cell of interest compared to the control cell indicates that the cell of interest expresses the OATP2A1.

25. The method of claim 16, wherein the cell overexpresses at least 100 times more OATP2A1 mRNA compared to a control cell.

26. The method of claim 16, wherein the cell is extracted from a mammalian tissue or human tissue.

27. The method of claim 16, wherein the compound comprises a new chemical entity (NCE), a nonsteroidal anti-inflammatory drug (NSAID), a modulator of eicosanoid receptor, a modulator of prostanoid receptor, or a modulator of prostaglandin transporter (PGT).

28.-30. (canceled)
